(19) United States
(12) Patent Application Publication
(10) Pub. No.: US 2014/0005607 A1
(43) Pub. Date: Jan. 2, 2014

(54) MULTI-LAYER MEDICAL DRESSINGS

(71) Applicant: Centurion Medical Products Corporation, Williamston, MI (US)

(72) Inventors: Tamer S. Elsamahy, Brighton, MI (US); Thomas Archipley, II, Howell, MI (US)

(21) Appl. No.: 13/932,343

(22) Filed: Jul. 1, 2013

Related U.S. Application Data
(60) Provisional application No. 61/666,304, filed on Jun. 29, 2012.

(51) Int. Cl. A61M 25/02

(52) U.S. Cl. CPC A61M 25/02
USPC 604/180

(57) ABSTRACT

A multi-layer dressing is disclosed. The dressing includes at least a primary layer comprised of a first layer, a transparent layer and a release liner. The first layer has an opening therethrough. The transparent layer is configured to cover the opening in the first layer. At least one of the first layer and the transparent layer includes a bottom adhesive surface. The release liner is attached to at least a portion of the bottom adhesive surface. The release liner is configured to provide an unobstructed view through the opening when the release liner is attached to the bottom adhesive surface. An optional closure piece is also disclosed, wherein the closure piece may be selectively altered to accommodate various sized catheter devise.

### FIELD OF TECHNOLOGY

The present disclosure relates to dressings, including multi-layer dressings used in medical applications. Such dressings may protect, secure, and/or allow viewing of catheters, introducer sheaths, infusion needles, implanted port Huber access needles, medical tubing, or the like.

### BACKGROUND

It is known to use dressings at or around insertion sites on a patient. Conventional dressings may have certain limitations. For example, in some conventional dressings, a portion of a folded release liner may block the view of an otherwise fully unobstructed transparent window, making proper positioning and application of the dressing more challenging for the medical professional.

Some conventional dressings have a size or shape that lacks the versatility to accommodate a wide variety of vascular access catheters (e.g., central venous catheters (“CYCs”), peripherally inserted central catheters (“PICCs”), ports, peripheral intravenous catheters (“PIVs”), sheaths, etc.), especially large bulky catheters with tubings, lumens, ports, etc., that emanate in various directions. In such conventional dressings the dressing often goes over the entire cluster of exiting lumens and tubings. Such arrangements cause undesired “tenting” where the dressing creates a tent over the bulky catheters thereby reducing the area of dressing in contact with skin. This situation also provides less protection for the insertion site. Additionally, the larger the tent created, the lower the dwell time or performance of the dressing, predominately due to a reduced tug force withstand.

Some conventional catheter care dressings include a center notch in their structure out of which clinicians may route and exit one or more of the lumens/tubings/pigtails. Doing so, however, may cause higher stresses on the notch. Such stresses may eventually overpower the adhesive, causing the dressing to peel away from the skin prematurely.

Some conventional dressings are accompanied by closure pieces, which may, by nature of their cooperative complementing design, reduce the stresses on the dressing that is securing the catheter(s) in place, Conventional closure pieces may have a single complementary notch only, thereby providing less versatility to be used with a variety of catheters configured with multi-lumen/multi-pigtails in a variety of sizes and shapes, and physical arrangements/directions of tubings exiting the dressing and of varying rigidity/flexibility.

It is desired to address one or more such limitations with the dressings disclosed herein.

### BRIEF DESCRIPTION OF THE DRAWINGS

The features and inventive aspects will become apparent upon reading the following detailed description, claims, and drawings, of which the following is a brief description:

FIG. 1 illustrates a top view of an exemplary multi-layer dressing assembly.

FIG. 2 illustrates an exploded view of the multi-layer dressing assembly of FIG. 1.

FIG. 3 is a cross-sectional view of the multi-layer dressing assembly of FIG. 1 taken along lines 3-3.

FIG. 4A illustrates a top plan view of a portion of the exemplary multi-layer dressing of FIG. 1.

FIG. 4B illustrates a bottom plan view of the portion of the exemplary multi-layer dressing of FIG. 4A, with an anchor release liner intact.

FIG. 4C illustrates a bottom plan view of the portion of the exemplary multi-layer dressing of FIGS. 4A-4B, with an anchor release liner removed.

FIG. 5 is an environmental view of a portion of the exemplary multi-layer dressing of FIG. 1 securing a multi-lumen/multi-pigtailed catheter.

FIG. 6 is another environmental view of the portion of the exemplary multi-layer dressing of FIG. 5 securing a multi-lumen/multi-pigtailed catheter.

FIGS. 7A-7C illustrate exemplary applications of a multi-layer dressing with an exemplary closure piece with a central region selectively removed.

FIG. 7D illustrates an exemplary application of a multi-layer dressing with an exemplary closure piece having a central region therein.

### DETAILED DESCRIPTION

Referring now to the drawings, illustrative examples are shown in detail. Although the drawings represent certain examples, the drawings are not necessarily to scale and certain features may be exaggerated to better illustrate and explain an innovative aspect of an example. Further, the examples described herein are not intended to be exhaustive or otherwise limiting to the precise form and configuration shown in the drawings and disclosed herein.

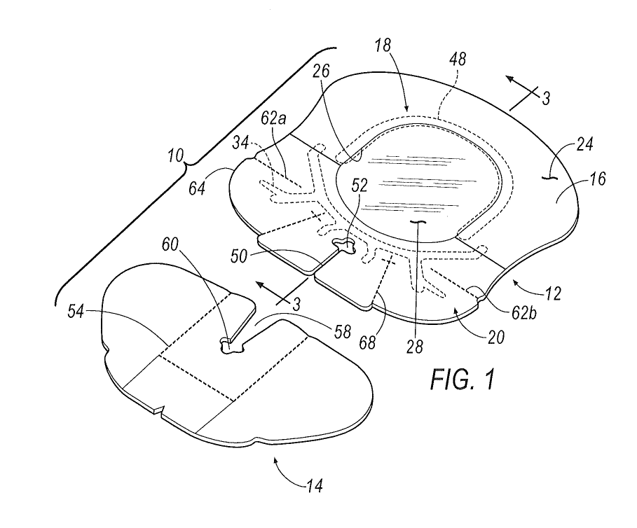

Referring to FIG. 1, an exemplary multi-layer dressing assembly 10 is disclosed. While the Figures illustrate an exemplary arrangement of a multi-layer dressing assembly, it is understood that many configurations of layers and stack ordering of layers and different numbers of layers may be suitable for use with the dressings disclosed herein. Thus, multi-layer dressing assembly 10 is not limited to the particular number of layers or stacking order shown in the Figures.

The multi-layer dressing assembly 10 may be used to view, protect, and secure a catheter inserted into a catheter insertion site such as a PICC (“peripherally inserted central venous catheter”), insertion site, a jugular insertion site, a subclavian insertion site, a femoral insertion site, or an implanted port insertion site. The multi-layer dressing assembly 10 is capable of securing a variety of sizes, shapes, and types of catheters (single lumen, double lumen, triple, and quad lumen), infusion needles, and associated hubs, ports, and tubing. The multi-layer dressing assembly 10 provides protection against microbial ingress and site or patient systemic infection, and secures the catheter and associated hubs, ports, and tubing so that forces acting on the tubing and catheter do not peel the dressing from a patient's skin or cause the catheter to become dislodged.

As shown in FIG. 1, in one exemplary configuration, multi-layer dressing 10 may comprise a primary dressing portion 12 alone, or a primary dressing portion 12 in combination with a closure piece 14 or other accessories and/or structures. Moreover, the multi-layer dressing may also include features disclosed in other patents or patent applications owned by the present assignee, Centurion Medical Products Corp., including, but not limited to, adhesive free zones (“AFZs”) and integrated anti-microbials, etc. Examples of such features may be found in U.S. Ser. Nos. 12/821,420; and 12/956,473, the contents of which are incorporated by reference in their entirety.

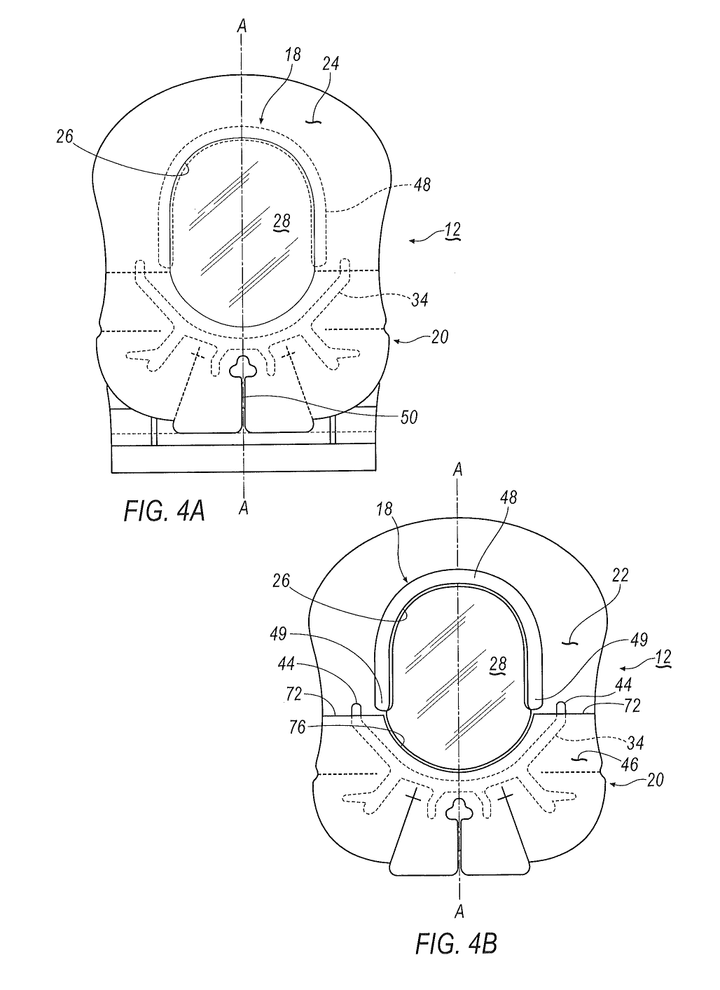

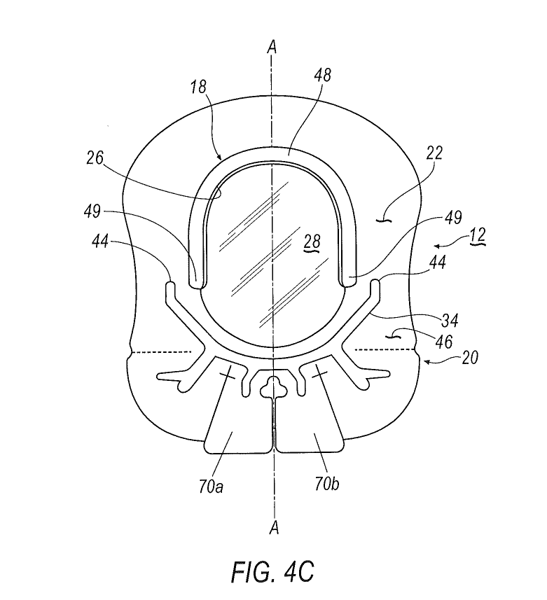

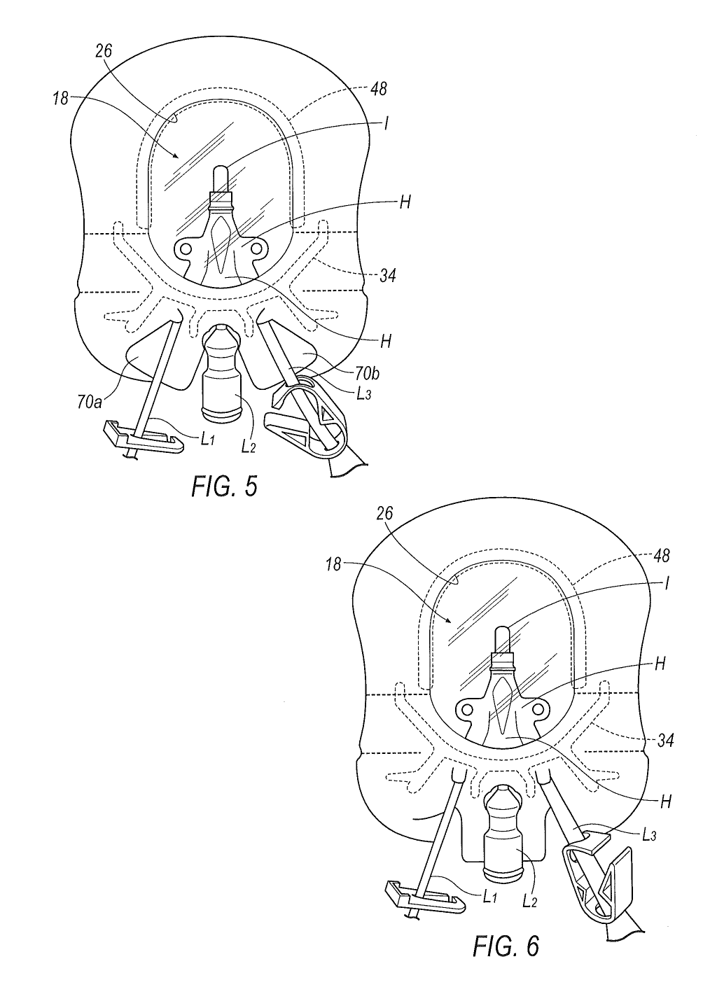

With reference to FIGS. 1 through 4, the primary dressing portion 12 of the multi-layer dressing assembly 10 includes a first layer 16 having an insertion site viewing portion 18 and an anchor member portion 20 that are juxtaposed. In the depicted exemplary configuration, the top layer may comprise any number of materials. For example, the first layer 16 may be a woven or non-woven material. Synthetic materials are also contemplated. The first layer 16 has an adhesive side 22 (best seen in FIGS. 4B and 4C) and an opposite non-adhesive top side 24. The adhesive side 22 may be coated with any suitable medical grade adhesive.

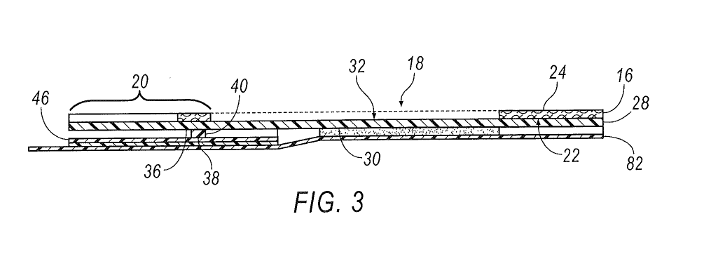

The insertion site viewing portion 18 is defined by an opening 26 in the first layer 16. In one exemplary arrangement, a transparent film layer 28 having an adhesive skin-adhering side 30 and an opposite non-adhesive side 32 (see FIG. 3) is adhered by its non-adhesive side to the fabric layer adhesive side 22. The transparent film layer 28 serves to close the opening 26 in the first layer 16. The transparent film layer 28 may be a polyurethane film coated on one side with any suitable medical grade adhesive. In some exemplary multi-layer dressings 10, the film layer 28 may further include adhesive over some or all of its skin-facing surfaces and may extend to the outer perimeter of dressing in all directions. Alternatively, (not shown) the transparent film layer 28 may not extend all the way to the outer edge of the first layer 16 such that the outer edge of the first layer 16 may extend beyond an outer edge of the transparent film layer 28. In some instances, an adhesive free zone (AFZ) directly over some or all of the insertion site 18 may be included. Such an arrangement is suitable to reduce adhesion to such items as antimicrobial biopatches, safety Huber needle mechanisms, etc. In another exemplary arrangement, the film layer 28 may be disposed on top of the first layer 16. In such an arrangement, the top surface of the film layer 28 will be non-adhesive, while at least a portion of the bottom surface has adhesive thereon.

The anchor member portion 20 may include an anchor member such as a reinforcing structure 34 disposed on the first layer 16. In one exemplary arrangement, the reinforcing structure 34 has a non-adhesive top surface 36 and a bottom adhesive surface 38. The top non-adhesive surface 36 is adhered to the first layer adhesive side 22 such that the reinforcing structure 34 is disposed on the bottom of the first layer 16. However, it is understood that the present disclosure is not limited to this arrangement. For example, the reinforcing structure 34 may be disposed on a top surface of the fabric layer 22, such as that disclosed in co-owned U.S. Pat. No. 8,212,101, the contents of which are incorporated by reference. The reinforcing structure 34 may be made of a polypropylene net material, a net-like material, or another similar material having rigidizing and force spreading properties as discussed below.

In an embodiment where the reinforcing structure 34 is on top of the first layer 16, a clinician or other user may easily view the reinforcing structure 34 for proper application of the dressing around a catheter, hub, and tubing on a patient's skin. Proper placement of the reinforcing structure 34 relative to the catheter hub and tubing provides maximum securement of the dressing assembly 10. Also, placement of the reinforcing structure 34 on top of the first layer 16 provides ease of manufacture. However, the present disclosure is not limited to this arrangement.

For example, as may be seen best in FIGS. 3 and 4B-4C, in one exemplary arrangement, the reinforcing structure 34 may be adhered to the skin-adhering side 32 of the transparent film layer 28. In this arrangement, when the dressing assembly 10 is positioned on a patient's skin, the reinforcing structure 34 will be in direct contact with the patient's skin.

The reinforcing structure 34 itself may also be provided with a distinctive color to aid in proper placement. For example, the non-adhesive surface 36 of the reinforcing structure 34 may include a colorant, or a colorant may be disposed on the adhesive surface 38 of the reinforcing structure 34. Alternatively, the reinforcing structure 34 may include a colorant therein. For example, the colorant may be directly added to the raw material from which the reinforcing structure 34 is made. The colorant improves the visibility of the reinforcing structure 34 and greatly aides in proper placement of the dressing assembly 10 on a patient's skin such that the reinforcing structure 34 is properly disposed relative to a catheter hub, tubing, etc. In one exemplary arrangement, the color of the reinforcing structure 34 may be visible through the first layer 16 to aid in proper placement.

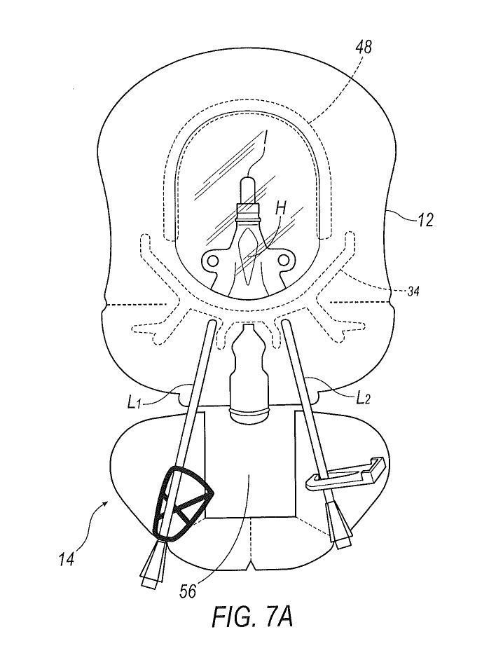

The reinforcing structure 34 may be any shape that has multiple axes such as an X-shape, another similar hub-and-spoke shape, or a backbone and rib shape. In one specific embodiment, the reinforcing structure 34 may include a central body 40 and a plurality of spaced ribs 42 extending outwardly from the central body 40. The central body 40 may be generally curved to at least partially follow the contour of the opening 26, with the ribs 42 extending radially outwardly away from the opening 26. Ends 44 of the central body 40 may extend beyond a bottom anchor release liner 46, as will be discussed below in further detail. As will also be discussed below in further detail, the reinforcing structure 34 is configured to be generally disposed across a catheter hub, as illustrated in FIGS. 5-7, for example. The reinforcing structure 34 may be generally symmetric about one of its axes. The central body 40 of the reinforcing structure 34 may be generally shield-shaped.

The reinforcing structure 34 strengthens the dressing assembly 10 by making the assembly less floppy for easier application to a patient's skin. More importantly, when the dressing assembly 10 is applied to a patient's skin, the reinforcing structure 34 spreads the external forces that are exerted on the dressing assembly 10 by the tubing over a large surface area, greatly increasing the dressing's resistance to premature separation from the patient's skin. Likewise, the reinforcing structure 34 increases the amount of force necessary to separate the dressing assembly 10 from a patient's skin. External forces are not as localized which is the typical reason small forces are able to commence peeling of a dressing by stretching the fabric and film in a local area which then propagates onward. Commonly, external forces are exerted on the dressing by pulling, snagging, or tugging on the ports, pigtails, fittings, and/or medical tubing that are connected to the catheter hub. For example, movement of medical tubing may be caused by the patient moving, by snagging of the tubing on other neighboring objects, by a clinician moving the tubing or the patient, or any combination of the above. The reinforcing structure 34 also prevents premature separation of the dressing assembly 10 from a patient's skin by preventing the dressing assembly 10 from stretching when the dressing assembly 10 is tugged on as described above, for example, when the tubing connected to the catheter hub is pulled on. Stretching of a dressing assembly 10 locally can ultimately lead to a dressing assembly 10 separating fully from a patient's skin. In sum, the reinforcing structure 34 increases the withstand of the dressing assembly 10 and greatly increases the amount of multi-directional pulling force that is necessary to cause the dressing assembly 10 to separate from a patient's skin.

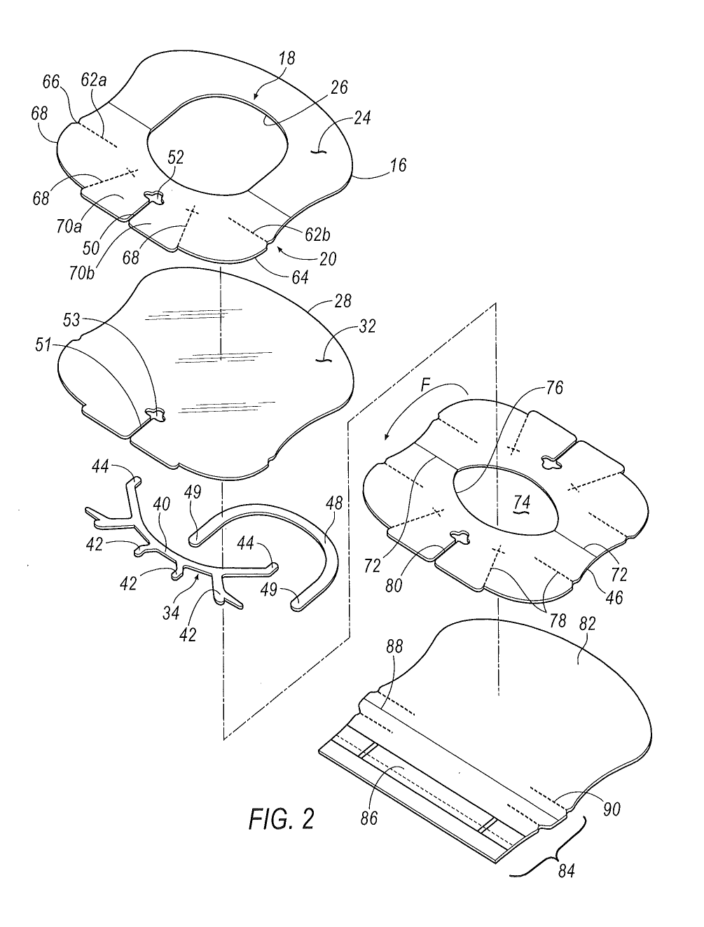

A pad member 48 may be adhered to the adhesive skin-adhering side 30 of the transparent film layer 28. The pad member 48 may at least partially circumscribe the viewing portion opening 26. In one exemplary arrangement, the pad member 48 is defined by ends 49 that are positioned above the bottom anchor release liner 46, as best seen in FIG. 4B. The pad member 48 may be relatively thin and may have a low absorbent capacity, such as a capacity of approximately 2-3 cc. The pad member 48 is comprised of an absorbent material and may provide a wicking function. Suitable materials include, but are not limited to a cellulose fiber, optionally laminated with a low adherent netting. As depicted in FIG. 2, the pad member 48 rests beneath a portion of the transparent film layer 28. Different shapes and sizes and layer arrangements of absorbent material may also be suitable. The pad member 48 may also include an integrated antimicrobial therein.

The pad member 48, however, is capable of preventing the egress of certain amounts of exudate and other liquids from beyond the viewing portion. It is not necessary for the pad 48 to have a large absorbent capacity for most typical catheters and insertion sites. It is likely that in practice the dressing would be replaced before or at the time that a small amount of exudate has been absorbed by the pad member 48.

A bottom slot 50 may extend inwardly from an edge of the first layer 16. The bottom slot 50 may be disposed in the anchor member portion 20 proximate the reinforcing structure 34. As will be explained below, the bottom slot 50 provides a location for tubing to exit from underneath the dressing assembly 10 and helps secure the tubing in place. A keyway 52 may be formed at an end of the bottom slot 50. In one exemplary arrangement, the keyway 52 has a generally clover shape, though other shapes of the keyway 52 are contemplated.

As shown in FIG. 2, the film layer 28 in certain embodiments where the film layer 28 extends toward the outer periphery of the first layer 16, is similarly provided with a bottom slot 51 that is configured to align with the slot 50 formed in the first layer 16. Bottom slot 51 may also terminate with a keyway 53 similar to keyway 52 of the first layer 16.

The closure piece 14 (best seen in FIG. 1) is configured to be applied at least partially over the primary dressing portion 12 for added stability. More specifically, the closure piece 14 may be secured across the bottom slot 50 and underneath the tubing and snug against the tubing, for increased securement of the dressing at the tubing exit, as will be discussed in further detail below.

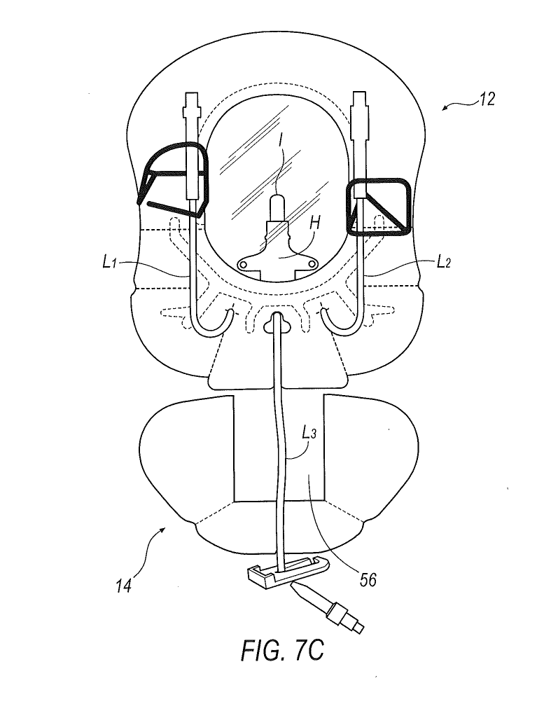

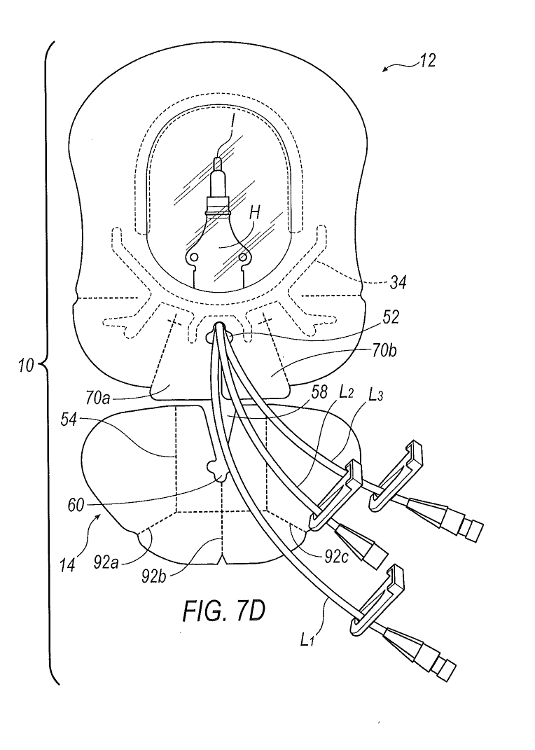

In one exemplary configuration, the closure piece 14 may be provided with a perforated box 54, as best seen in FIG. 1 (and further discussed below in more detail in connection with FIGS. 7A-7D). The closure piece 14 may be selectively modified by tearing the closure piece 14 about the outline of the perforated box 54 to create a wide U-shaped notch 56 (best seen in FIGS. 7A-7C) configured to permit exiting of various lumens/tubings/pigtails. Within the perforated box 54, however, a top slot 58 may extend inwardly from an outside edge of the closure piece 14. In one exemplary arrangement, the top slot 58 is configured to taper outwardly toward the edge of the closure piece 14 to allow for ease of positioning the closure piece 14 around any tubing or catheter hubs. The end of the top slot 58 may also be provided with a keyway 60. In one exemplary arrangement, the keyway 60 has a generally clover shape. The top slot 58 is configured to cooperate with bottom slot 50 to secure around smaller tubing when the perforated box 54 is kept intact. This configuration of the closure piece 14 therefore provides greater flexibility for the clinician in securing various catheter devices.

The dressing assembly 10 may further include a pair of side perforation lines 62a, 62b. Each side perforation line 62a, 62b extends inwardly from an edge 64 of the first layer 16 in the anchor member portion 20 as well as any film layer 28 in that region. The side perforation lines 62a, 62b may be generally perpendicular to the first layer 16 edge. A landmark notch 66 may be disposed along the fabric layer edge 64 at an end of each side perforation line 62a, 62b. The landmark notches 66 aid a clinician or other user in locating the ends of the side perforation lines 62a, 62b when it is necessary or desirable to tear one of the side perforation lines 62a, 62b. The side perforation lines 62a, 62b may be separated and opened prior to application of the dressing assembly 10 to more effectively anchor tubing extending from sideported catheter hubs such as a sideported CVC. Each of the pair of side perforation lines 62a, 62b may be disposed symmetrically on opposite sides of the dressing assembly 10, allowing the dressing assembly 10 to accommodate either left-handed or right-handed sideports although such a configuration is optional.

The primary dressing portion 12 further comprises one or more additional slits or perforations 68. The slits 68 cooperate with bottom slot 50 to create one or more movable flaps 70a, 70b. Landmark notches 69 may also be disposed along the fabric layer edge 64 at an end of each slit 68 to assist in locating the slit 68. The flaps 70a, 70b may be moved to accommodate most of the shapes, sizes, configurations, geometry of most commercially available vascular catheters requiring securement at or near an insertion site. For example, with the movable flaps 70a, 70b, the multi-layer dressing has improved ability to secure medical devices including, but not limited, to vascular access devices (“VADs”) such as peripheral intravenous catheters (“PICVs”), peripherally inserted central catheters (“PICCs”), central venous catheters (“CVCs”), dialysis, introducer sheaths, and swan-ganz catheters. The movable flaps 70a, 70b may include at least a portion of the reinforcing structure 34 thereon. More specifically, in some exemplary multi-layer dressings, the movable flaps 70a, 70b include a rib 42 of reinforcement material extending from the central body 40. The rib 42 thus may extend at least partially into the movable flap 70a, 70b. With this configuration, the reinforcement structure 34 in the movable flaps 70a, 70b may improve ease of handling, prevent tangling, and/or provide stability in the movable flaps 70a, 70b after they are moved from their original position of manufacture. The slits 68 may be performed, or perforations may be provided in the dressing assembly 10. With this configuration, the dressing assembly 10 may be selectively torn along the perforations to create the slits 68, thereby facilitating a desired positioning of the dressing assembly 10.

Dressing assembly 10 further comprises one or more release liners. In one exemplary configuration, dressing assembly 10 includes the bottom anchor release liner 46. The bottom anchor release liner 46 covers at least a portion of the adhesive skin-adhering side of the film layer 28. In one example, as shown in FIG. 2, the anchor release liner 46 may include a fold line 72 that permits the anchor release liner 46 to be folded upon itself as indicated by arrow F. In this arrangement, the anchor release liner 46 includes a central opening 74. When the anchor release liner 46 is folded upon itself, as shown in FIG. 4B, the central opening 74 forms a curved edge 76 that generally follows the contour of the opening 26 that defines the insertion site viewing portion 18. In this manner, the anchor release liner 46, due to the curved edge formed by the folded over opening 26, does not obstruct any part of the insertion site viewing portion 18 during application to a patient's skin. The anchor release liner 46 may also be provided with slits or perforations 78 and a slot 80 that generally correspond to the slits/perforations 68 and bottom slot 50 formed in the first layer 16 (as well as the slot 51 formed in the film layer 28. In this manner, the dressing assembly 10 may be properly positioned about one or more catheter devices before the anchor release liner 46 is removed from the dressing assembly 10 and the dressing assembly 10 is secured to a patient's skin. The anchor release liner 46 is configured to symmetrical about the fold line 72. The release liner may comprise any of a number of materials, uncoated paper and paper coated at least partially with, for example, a silicone-containing material. In another exemplary embodiment, the anchor release liner 46 need not be a folded member. Instead, the anchor release liner 46 may only extend to the fold line 72.

A second release liner 82 may also be employed to cover the remainder of the primary dressing's 16 exposed adhesive surface area 22. In one exemplary arrangement, the second release liner 82 may cover the adhesive skin-adhering side 30 of the insertion site viewing portion 18. The second release liner 82 prevents the adhesive on the film layer 28 from inadvertently and prematurely sticking to an object prior to application. In a specific embodiment, the second release liner 82 may be a single piece release liner with a lower portion 84 that extends beyond the anchor member potion 20. The lower portion 84 may also include a selectively removable data sheet 86 where information, for example the gauge of the catheter, the date/time that the dressing assembly 10 was last applied, as well as the initial of the clinician who applied the dressing assembly 10, may be recorded. A fold line 88 may be provided to facilitate controlled removal of the release liner 82 from the dressing assembly 10. Slits or perforation lines 90 may also be provided in the second release liner 82 to permit selective placement of the dressing assembly 10. The perforation lines 90 may be aligned with corresponding slits 62a, 62b and 78 formed in the first layer 16 and anchor release liner 46, respectively.

The primary dressing portion 12 (as well as the closure piece 14) may be symmetrical about a longitudinal axis A-A that extends through the insertion site viewing portion 18 and the anchor member portion 20, and splits the primary dressing 12 into equivalent left-hand and right-hand sides (see, e.g., FIG. 4A). The symmetry of the dressing 10 allows the dressing to be used on either the left-hand or right-hand side of a patient's body. However, it is understood that other geometries of the dressing may deviate greatly from this specific embodiment and will accomplish similar tug withstand performance.

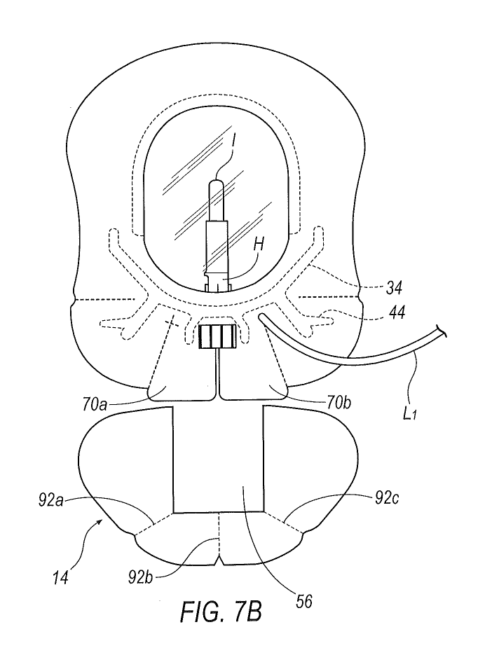

Referring to FIGS. 5-6, certain elements of the dressing assembly 10 will be explained in further detail. More specifically, to apply the dressing assembly 10 to an insertion site on a patient, the portion of the second release liner 82, which covers the insertion site viewing portion 18 of the dressing assembly 10 is peeled back to expose the adhesive skin-adhering side 30 on the transparent film layer 28. The insertion site viewing portion 18 is centered about a catheter insertion site I on a patient so that the insertion site I is generally in the center of the opening 26. Because the bottom anchor release liner 46 is folded about its opening 74 along fold line 72, the contour of the folded bottom anchor release liner 46 does not obstruct the clinician's view while the placing the dressing assembly 10 on the patient. Such an arrangement therefore allows for greater accuracy in proper positioning of the dressing assembly 10. Further, because the opening 26 is generally positioned so as to place the insertion site I at the center of the opening 26, this arrangement allows the insertion site Ito be easily viewed and monitored through the opening 26.

At the same time, an end of a catheter hub H may be partially viewed through the opening 26, as well as being partially positioned underneath the central body 40 of the reinforcing structure 34. The properly aligned insertion site viewing portion 18 is then adhered to the patient's skin. However, the bottom anchor release liner 46 is still adhered to the anchor member portion 20 of the dressing assembly 10. Accordingly, once the insertion site viewing portion 18 is adhered, a portion of the anchor release liner 46 that is folded under a portion of the anchor release liner 46 that is adhered to the anchor member portion 20 may be gripped and pulled away from the primary dressing member 12. This action serves to unfold the anchor release liner 46 about the fold line 72 until the anchor release liner 46 releases from the skin-adhering side 30 of the film layer 28 such that the skin-adhering side 30 is secured to the patient's skin.

Lumens L₁-L₃ that are operatively connected to the hub H are generally arranged to exit from underneath the primary dressing 10. However, vascular access devices such as peripheral intravenous catheters, peripherally inserted central catheters, central venous catheters, and dialysis catheters all vary in size and shape, as well as vary in the number of lumens or pig tails present for each catheter type or style. As a result of this variation, bulky and irregular catheter arrangements tend to lead to stresses or forces exerted by the catheter hubs and/or lumens on the dressing assemblies 10. Such stresses and forces are a main contributor to dressing assemblies 10 inadvertently lifting off of the patient's skin. To address this issue, the slits/perforations 68 cooperate with the top slot 58 to define selectively adjustable flaps 70a, 70b.

As best seen in FIGS. 5 and 6, selectively adjustable flaps 70a, 70b have been formed by tearing along the perforations in slits 68. The resulting flaps 70a, 70b may then be moved from their original position of manufacture and maneuvered around catheter hubs and/or catheter lumens/extension/pigtails, etc. L₁-L₃, or any of a number of other VAD elements. These flaps 70a-70b allow the primary dressing 12 to “contour” over bulky and irregular catheter arrangements, thereby lowering any stresses exerted on the primary dressing 12. Further, the slits 68 that create the movable flaps 70a-70b may also serve as an exit point for VAD lumens/tubings/extensions.

The use of the slits/perforations 68 of the primary dressing 12 also reduce the “tenting” phenomenon, whereby a portion of the dressing assembly 10 does not adhere to the skin. Tenting is especially an issue for applications that utilize larger hub sizes, because as the hub size increases, the larger the tent opening will be created. This issue will reduce dwell time and dressing assembly 10 performance. Because the slits 68 permit the flaps to contour to the particular VAD element, tenting is greatly reduced.

In one exemplary arrangement, in addition to providing reinforcement for the hub H, the reinforcing structure 34 also serves to ensure that the flaps 70a, 70b do not get inadvertently tangled onto themselves or to the dressing assembly 10. More specifically, some of the ribs 42 are configured to partially extend into the area where of the first layer 16 where flaps 70a, 70b are formed, as best seen in FIG. 4C.

Referring to FIGS. 7A-7D, various exemplary applications of a multi-layer dressing assembly 10 are shown on at least (4) different manufacturers configuration of catheters and introducer sheaths. In these examples, the primary dressing member 12 cooperates with the closure piece 14, as will be explained. The exemplary closure piece 14, as well as primary dressing member 12, includes a series of openable perforations, 92a-92c, permitting one or more regions to be removed from the closure piece 14 or just opened, as needed by a clinician. For example, as shown in FIGS. 7A-7C, the perforated box 54 of the closure piece 14 is removed to selectively form a U-shaped notch 56. Once removed, the closure piece 14 is moved over top of the primary dressing member 12 to aid in securing the VAD elements and the dressing assembly 10 in place. More specifically, portions of the closure member 14 overlay the anchor member portion 20, but permitting the lumens and other VAD elements to overlay the closure member 14. Generally, where the slits 68 in the primary dressing member 12 are used as an exit point for lumens/tubings/pigtails L₁-L₃, it may be advantageous to remove the perforated box 54.

However, it is also contemplated that there may be situations or VAD assemblies where it the perforated box 54 should be kept intact. For example, in reviewing FIG. 7D, it may be seen that the hub H has all three lumens L₁-L₃ exiting in the same direction. The lumens L₁-L₃ exit the primary dressing member 12 through the keyway 52. In this instance, the perforated box 54 will be kept intact, and the top slot 58 in the closure piece 14 will be fitted around the lumens L₁-L₃ as the closure piece 14 is overlaid onto to the primary dressing member 12. In this manner, the perforated box 54 of the closure piece 14 will provide additional resistance against the forces tending to cause the dressing assembly 10 to peel off the patient's skin.

Because both the primary dressing member 12 and the closure piece 14 are versatile in how the pieces may be positioned around a plurality of different catheter arrangements, no matter what type of catheter device is employed, the dressing assembly 10 may be able to contour itself around various components without forcing the lumens L₁-L₃ to take different shapes. Moreover, tenting and accidental dislodgement of the dressing assembly 10 is at least minimized, if not avoided.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

All terms used in the claims are intended to be given their broadest reasonable constructions and their ordinary meanings as understood by those skilled in the art unless an explicit indication to the contrary in made herein. In particular, use of the singular articles such as “a,” “the,” “said,” etc. should be read to recite one or more of the indicated elements unless a claim recites an explicit limitation to the contrary.

1. A multi-layer dressing, comprising:
a first layer having an opening therethrough;
a transparent layer configured to cover the opening in the first layer;
wherein at least one of the first layer and the transparent layer includes a bottom adhesive surface; and
a release liner attached to at least a portion of the bottom adhesive surface, wherein the release liner is configured to provide an unobstructed view through the opening when the release liner is attached to the bottom adhesive surface.

2. The multi-layer dressing of claim 1, further comprising a reinforcing element operatively connected to the first layer.

3. The multi layer dressing of claim 2, wherein the first layer includes a viewing portion and an anchor portion, wherein the opening is formed in the viewing portion and wherein the reinforcing element is disposed on the anchor portion.

4. The multi-layer dressing of claim 1, further comprising pad member, wherein the pad member is secured to the bottom adhesive surface such that the pad member is configured to contact a patient's skin.

5. The multi-layer dressing of claim 4, wherein the pad member is disposed at least partially about the opening in the first layer.

6. The multi-layer dressing of claim 5, wherein the pad member includes an anti-microbial.

7. The multi-layer dressing of claim 1, wherein a top surface of the transparent layer is adhered to a bottom adhesive surface of the first surface, wherein a bottom surface of the transparent layer is an adhesive surface, and wherein the release liner is secured to the bottom surface of the adhesive surface.

8. The multi-layer dressing of claim 7, wherein the release liner is defined by a body member bisected by a fold line and having an opening therein, wherein the body member is folded on itself about the fold line to create a contoured edge that generally corresponds with an edge of the opening.

9. The multi-layer dressing of claim 1, further comprising an exit slot positioned adjacent the window.

10. The multi-layer dressing of claim 9, further comprising at least one perforated slit extending inwardly from an edge of the dressing so as to be adjacent the slot, the slit and the slot cooperating to define a moveable flap that may selectively adjusted to accommodate a plurality of medical devices of different shapes and sizes extending from the dressing.

11. The multi-layer dressing of claim 10, wherein the movable flap comprises a reinforcement material.

12. The multi-layer dressing of claim 10, further a perforated slit disposed on either side of the slot.

13. The multi-layer dressing of claim 1, further comprising a secondary release liner configured to attach to a portion of the bottom adhesive surface and cover the release liner that is not obstructing the opening.

14. The multi-layer dressing of claim 1, wherein the further comprising a separate closure piece configured to selectively overlay a portion of the first layer.

15. The multi-layer dressing of claim 14, wherein the closure piece further comprises at least one perforated section that permits selective alteration of the closure piece to accommodate medical devices of different shapes and sizes extending from the dressing.

16. The multi-layer dressing of claim 1, further wherein the bottom adhesive surface includes an adhesive free zone.

17. The multi-layer dressing of claim 2, further comprising pad member that is disposed at least partially about the opening in the first layer, wherein the pad member is secured to the bottom adhesive surface such that the pad member is configured to contact a patient's skin.

18. The multi-layer dressing of claim 2, wherein the reinforcing element is adhered to the bottom adhesive surface.

19. The multi-layer dressing of claim 18, wherein the reinforcing element includes a bottom adhesive surface.

20. The multi-layer dressing of claim 2, wherein the reinforcing element is colorized.
